(19) United States
(12) Patent Application Publication
(10) Pub. No.: US 2006/0036135 A1
(43) Pub. Date: Feb. 16, 2006

(54) Skin cancer identification template

(76) Inventor: Kenneth A. Kern, West Hartford, CT (US)

Correspondence Address: PEPE & HAZARD, LLP, 225 ASYLUM ST., HARTFORD, CT, 06103, US

(21) Appl. No.: 10/915,102

(22) Filed: Aug. 10, 2004

(51) Int. Cl. A61B 5/00

(52) U.S. Cl. 600/300

(57) ABSTRACT

A template for comparison of a skin blemish with the appearance of a multiplicity of different types of skin cancers has a lightweight panel with an aperture therethrough adapted to be disposed over a skin blemish, and a multiplicity of representations of different types of skin cancers on the panel and disposed about the aperture to enable facile comparison. A lens element may extend over the aperture to magnify the blemish. The lens or other transparent element extending over the aperture may have scale graduations thereon.

### BACKGROUND OF THE INVENTION

The present invention relates to a template for visually comparing skin lesions with photographic reproductions of different types of skin cancer.

There are many types of skin lesions, some of which are cancerous or precancerous. A skilled dermatologist or oncologist may have sufficient familiarity with the appearance of the different types of skin lesions to visually identify the particular type being evaluated. Physicians having less familiarity with lesions may consult reference works with photographic reproductions of various types of lesions in order to effect such identification. In either case, the identification is the first step in developing a course of further examination and treatment.

Obviously, searching through medical texts for color photographs of skin lesions while examining a lesion on the patient's body is not conducive to patient confidence or optimum utilization of the physician's time.

Various items have been provided to facilitate examination of skin lesions by a physician such as rulers, color guides and like devices which do not provide a means for facile identification of the type of lesion.

It is an object of the present invention to provide a novel template enabling side-by-side comparison of a skin lesion with color photographs of known types of skin cancers and other lesions.

It is also an object to provide such a template which can be readily manufactured at relatively low cost.

A further object is to provide such a template which may be readily placed over a subject lesion located on substantially any portion of the body.

### SUMMARY

It has now been found that the foregoing and related objects may be readily attained in a template for comparison of a skin blemish with the appearance of a multiplicity of different types of skin cancers and comprising a lightweight panel having an aperture therethrough and adapted to be disposed over a skin blemish. The template has a multiplicity of representations of different types of skin cancers on the panel disposed about the aperture to enable facile visual comparison.

Conveniently, the aperture is circular and the representations are spaced thereabout. The panel is substantially opaque, and the representations are photographs of different forms of skin cancers.

The panel may be a synthetic resin sheet material or a laminate of paper stock and transparent synthetic film. For providing a larger group of illustrations, a set of templates may be employed.

A measuring scale may be provided along one side of the panel.

In one embodiment, a lens element extends over the aperture to magnify the blemish, and the lens may have scale graduations thereon.

Alternatively, a non-magnifying transparent element may extend over the aperture and have scale graduations thereon.

### BRIEF DESCRIPTION OF THE DRAWINGS

FIG. 1 is a plan view of a template embodying the present invention;

FIG. 2 is a cross sectional view thereof with the thickness exaggerated;

FIG. 3 is a fragmentary perspective view of the arm of a patient with the template placed over a skin blemish being evaluated;

FIG. 4 is a plan view of another embodiment of the template; and

FIG. 5 is a cross sectional view of the embodiment of FIG. 4.

### DETAILED DESCRIPTION OF THE ILLUSTRATED EMBODIMENT

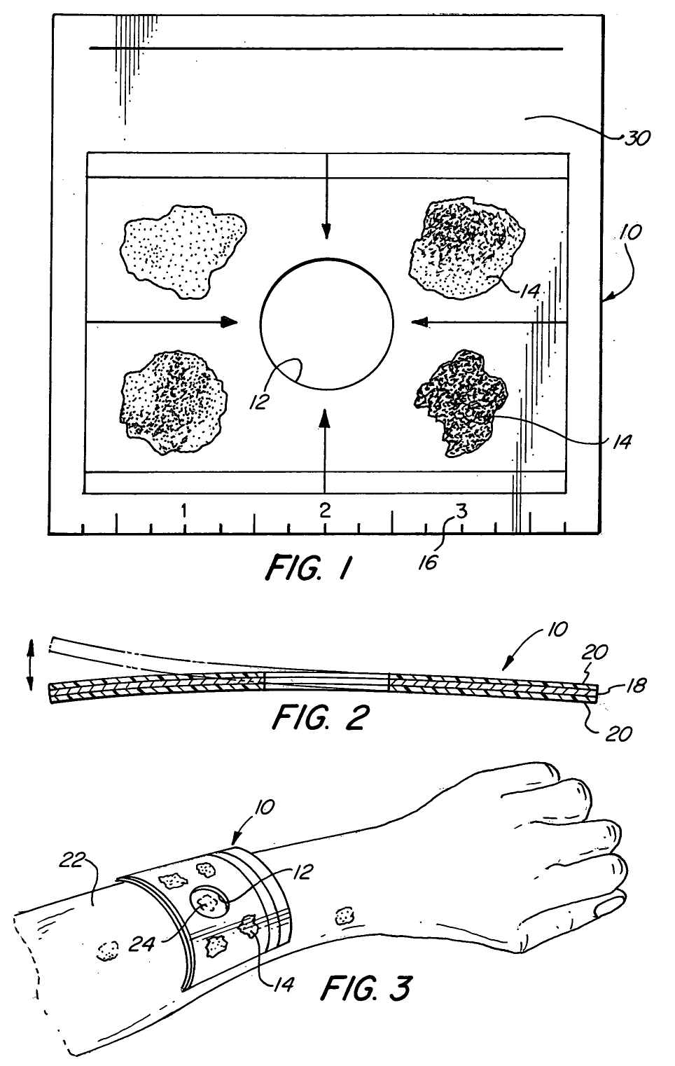

Turning first to FIGS. 1 and 2, therein illustrated is a template embodying the present invention and generally designated by the numeral 10. The template 10 has a central aperture 12 and a series of photographic reproductions 14 spaced about the aperture 12. Along one side of the template 10 is a measurement scale 16 which can be used to measure the size of a lesion being evaluated.

As seen in FIG. 2, the template is flexible and is conveniently a laminate of a paper core 18 upon which the photographs 14 are reproduced and plastic film layers 20.

As seen in FIG. 3, the template 10 is placed on the arm 22 of a patient so that a lesion 24 to be evaluated is located within the aperture 12. The physician can readily compare the lesion 24 with the photographic reproductions 14 of different types of cancer adjacent thereto to classify the lesion. The physician may also measure the size of the lesion by use of the scale 16.

As will be appreciated, a set of templates may be provided to allow comparison of a lesion with photographic reproductions of more types of cancers. In one embodiment, different photographic reproductions may be provided on both sides of the template.

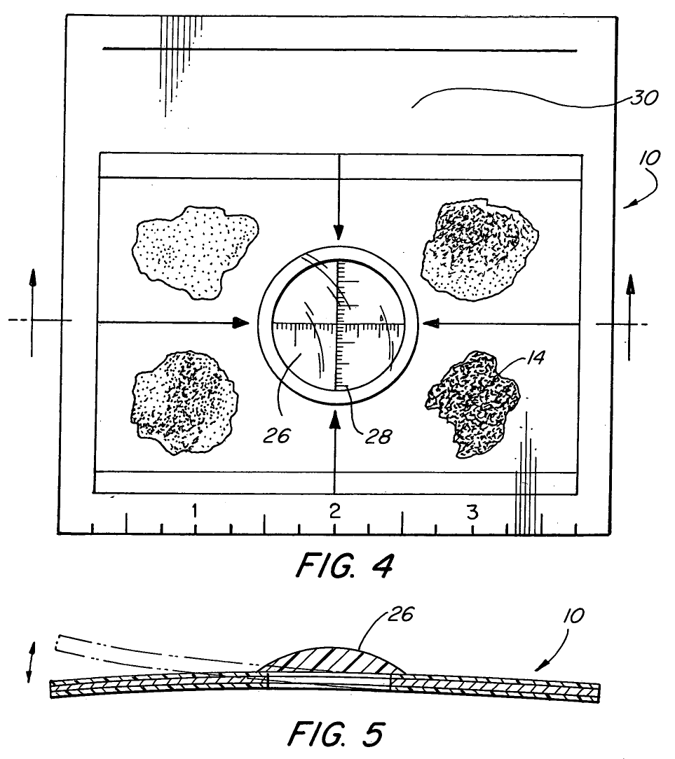

Turning next to FIGS. 4 and 5, this embodiment has a magnifying lens 26 adhered to one surface of the template 10a and extending over the aperture 12. This enables magnification of the lesion to facilitate identification and evaluation. The lens 26 may have graduations 28 thereon.

In both of the illustrated embodiments, the template 10 has a header portion 30 upon which instructions and information may be imprinted to facilitate use by the physician.

It will be readily appreciated that the panel of the template may be formed from materials other than the paper/plastic laminate so long as it retains the desired flexibility. For example, the photographic reproductions can be directly imprinted on a plastic sheet material, or photographic paper imprinted with the photographs and can be utilized without a coating of plastic material. Moreover, a coating may be applied as a liquid as well as a plastic film.

In the illustrated embodiment, the photographs have been reproduced on only one side of the template. However, additional photographic reproductions of other types of lesions may be imprinted on the reverse side so that a larger group of lesions is available for comparison. Moreover, lastly, sets of two or more templates and reproductions of different types of lesions may be provided to enable comparison with a still greater collection of known types of lesions.

It would be readily appreciated that the side-by-side comparison provided by the templates of the present invention provides a facile means for identification of the type of lesion being evaluated and minimizes the possibility of misidentification. The flexibility of the template enables it to be placed on the human body at almost any location since it can be readily formed about the subject surface.

Thus, it can be seen from the foregoing detailed specification and attached drawings that the template of the present invention which is simple and convenient to use and which is readily manufactured at relatively low cost.

Having thus described the invention, what is claimed is:

1. A template for comparison of a skin blemish with the appearance of a multiplicity of different types of skin lesions comprising:
(a) a lightweight panel having an aperture therethrough adapted to be disposed over a skin lesion; and
(b) a multiplicity of representations of different types of skin lesions on said panel and disposed about said aperture to enable facile comparison.

2. The template in accordance with claim 1 wherein said aperture is circular and wherein said representations are spaced thereabout.

3. The template in accordance with claim 1 wherein said panel is substantially opaque.

4. The template in accordance with claim 1 wherein said representations are photographs of different types of skin cancers.

5. The template in accordance with claim 1 wherein said panel is a synthetic resin sheet material.

6. The template in accordance with claim 1 wherein said panel is a laminate of paper stock and transparent plastic film.

7. The template in accordance with claim 1 wherein there is included a measuring scale along one side of the panel.

8. The templates in accordance with claim 1 wherein there is included a lens element extending over said aperture to magnify the lesion.

9. The templates in accordance with claim 8 wherein said lens has scale graduations thereon.

10. The templates in accordance with claim 1 wherein there is included a transparent element extending over said aperture and having scale graduations thereon.

11. A set of templates for comparison of a skin lesion with the appearance of a multiplicity of different types of skin lesions, each of said templates comprising:
(a) a lightweight panel having an aperture therethrough adapted to be disposed over a skin lesion; and
(b) a multiplicity of representations of different types of skin lesions on said panel and disposed about said aperture to enable facile comparison.

12. A set of templates in accordance with claim 11 wherein said panel is substantially opaque and said representations are photographs different types of skin cancers.

13. A set of templates in accordance with claim 11 wherein said aperture is circular and wherein said representations are spaced thereabout.

14. A set of templates in accordance with claim 11 wherein said panel is a laminate of paper stock and transparent plastic film.

15. A set of templates in accordance with claim 11 wherein there is included a measuring scale along one side of the panel.

16. A set of templates in accordance with claim 11 wherein
there is included a lens element extending over said aperture to magnify the lesion.

17. A set of templates in accordance with claim 16 wherein said lens has scale graduations thereon.

18. A set of templates in accordance with claim 11 wherein there is included a transparent element extending over said aperture and having scale graduations thereon.
